(19) United States
(12) Patent Application Publication
(10) Pub. No.: US 2006/0100430 A1
(43) Pub. Date: May 11, 2006

(54) Process for preparation of 2-alkoxy-6-(trifluoromethyl)pyrimidin-4-ol

(76) Inventors: Beat Schmidt, Baltschieder (CH); Laurent Ducry, Sierre (CH)

Correspondence Address: FISHER, CHRISTEN & SABOL, 1725 K STREET, N.W., SUITE 1108, WASHINGTON, DC, 20006, US

(21) Appl. No.: 10/543,777

(22) PCT Filed: Feb. 2, 2004
(86) PCT No.: PCT/EP04/00932

§ 371 Date: Oct. 7, 2005

(30) Foreign Application Priority Data
Jan. 31, 2003 (EP) 03001983.0

(51) Int. Cl. C07D 239/52

(52) U.S. Cl. 544/309

(57) ABSTRACT

A process for preparing a 2-alkoxy-6-(trifluoromethyl)pyrimidine-4-ol of the formula I: 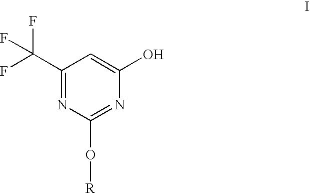
wherein R is C₁-C₈ alkyl. First a cyanogen halide, that is, cyanogen chloride ClCN or cyanogen bromide BrCN, is reacted with a C₁-C₈ alcohol ROH and/or a C₁-C₈ alcoholate in the presence of a base to give the corresponding symmetric imidocarbonic acid dialkylester of the formula II: 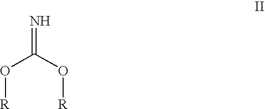 In a second step, the imidocarbonic acid diakylester is reacted with trifluoromethyl acetoacetic acid ethyl ester in the presence of ammonia to yield the compound of formula I.

The subject of the present application is a novel process for preparation of 2-alkoxy-6-(trifluoromethyl)pyrimidin-4-ol.

U.S. Pat. No. 5,717,096 of the same applicant as of the present invention teaches a process for preparation of the title compound. 2-alkoxy-6-(trifluoromethyl)pyrimidin-4-ol is an important compound used in the manufacture of acarizidal agents. Due to widespread use of acarizides in sanitation, efficient processes for manufacturer of raw materials are important for allowing for wide-spread application. The process disclosed in US'096 devises reacting cyanamide with an alcohol under acidic conditions to yield a corresponding alkoxyisourea hydrochloride which is then further converted to the the 2-alkoxy-6-(trifluoromethyl)pyrimidin-4-ol under conditions of base-catalysis. The last reaction step is carried out in the presence of an acetoacetate in aequeous solution in the presence of sodium hydroxide. The total yields range from 52% to 69%.

As a disadvantage, the process requires careful neutralization of the reaction broth in between reaction steps one and two. First the reaction mix needs to be cooled down at least to ambient temperature prior to neutralization. At larger scale, the neutralization reaction further presents a problem of heat transfer. Accordingly, proceeding from step 1 to step 2 is very time consuming and may negatively affect yield. The yield of the process was not entirely satisfactory and difficult to control.

The object of the present invention was to devise another process that lacks said disadvantages. This object was solved by a process according to independent claim 1.

A process according to the present invention for the preparation of a 2-alkoxy-6-(trifluoromethyl)pyrimidine-4-ol of formula I: 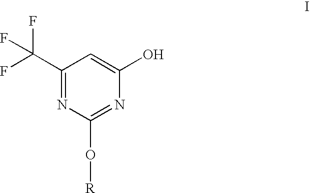
wherein R is a C1-C6-alkyl group, comprising, in a first step, reacting a cyanogen-halide selected from the group consisting of cyanogen-chloride C1-CN and cyanogen-bromide Br—CN, with a C1-C8 alcohol R—OH and/or an respective C1-C8 alcoholate in the presence of a base to give the corresponding symmetric imidocarbonic acid di-alkylester of formula II: 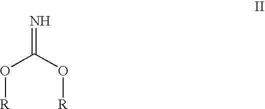
, and in a second step further reacting said imidocarbonic acid di-akylester with trifluoro-methyl-acetoacetic acid ethyl ester or any other C₁-C₆ alkyl ester thereof to compound I in the presence of ammonia.

Suitable trifluoromethyl-acetoacetic acid C₁-C₆ alkyl ester are e.g. methyl, ethyl, propyl, isopropyl, n-butyl, isobutyl, sek.-butyl, tert-butyl, pentyl, heptyl, hexyl esters. Preferably, said esters are methyl, ethyl, n-propyl, isopropyl, isobutyl or butyl.

Preferably, the cyanogenhalide is added to the reaction mixture at a temperature of below 20° C.

Preferably the product of formula II is obtained only by removing salt precipitates from the reaction broth of the first step, preferably removing the salt precipitates at a temperature <10° C. The resulting crude product is sufficiently pure to allow for optimal yields in the second reaction step. Removal is e.g. quickly and efficiently attained by filtration, streamlining the entire process. This embodiment may be more preferably combined with operating the entire reaction as a one pot reaction, that is using the alcoholic solution comprising the product of formula II from which solution the solid salt was removed directly for the second reaction step yielding the product of formula I. The alcohol may serve both as reagent and solvent for the first reaction step.

Another possible work-up method can be to add water to the reaction sump, allowing of separation of aequeous and non-aequeous phases for traditional working-up. It is also possible to use the reaction broth directly without any further isolation/work-up for the next reaction step. Such straightforward one-pot reaction sequence is another preferred embodiment of the present invention.

Preferably the second reaction step is carried out with 1.5 to 3 mol equivalents of ammonia

Preferably the second reaction step is carried out in an aprotic, polar solvent. Such solvent such as the alcohol employed for the first reaction step, or a suitable mixture of such solvent along with the alcohol of the first reaction step, ensures solubility of reagents whilst preventing base-catalyzed hydrolytic side reactions. Some amount of water can be introduced by the ammonia if expediently provided in aequeous solution. More preferably, the second reaction is carried out as a one pot reaction as described above and further employing the alcohol reagent of the first step as a solvent. Most preferably, the second reaction is carried out in isopropyl alcohol.

Preferably, the second reaction step is carried out at a temperature of from 50 to 100° C., preferably of from 60 to 90° C. Is goes without saying that the choice of the alcohol reagent which is expediently providing the solvent for the reaction may have a boiling point within those preferred temperature ranges and may therefore limit the maximally applicable temperature, requiring refluxing the solvent during reaction. More preferably, the second reaction step is carried out at two timely ordered temperature intervals, the first interval having a temperature below 65° C. in the preferred range and the second one having a temperature above 70° C. in the preferred range.

Preferably, the product compound of formula I is purified from the reaction sump by first removing the solvent and secondly crystallizing the compound of formula I from aequeous solution. More preferably, the pH is controlled at pH 5-7 during the crystallization step. Crystallization from water after removing the alcohol which is both solvent and reagent allows of instantaneous recovery of pure product (purity >98% as determined by HPLC). Further, water is optimal with regard to environmental concerns. Expediently, about 10 times the volume of the reaction sump in water are added after removal of alcohol.

In another preferred embodiment, the product compound of formula I is purified from the reaction sump by extraction with methylcyclohexene, crystallizing the product compound from the organic phase.

If an alcoholat is used, such alcoholat salt can be employed in quantitative amount in the presence of a suitable inert solvent such as an alcohol, preferably a secondary or tertiary alcohol, or it can be employed in substoechiometric or catalytic amounts in the presence of an alcohol as defined which alcohol is reacting with the cyanogenhalide and is solvent, too.

Preferably, the alcohol or alcoholat according to the present invention is an C1-C8 alkyl alcohol, preferably a C3-C5 alkyl alcohol. This is to be understood as to amount to the fact that the reaction is carried out free from any addition of an alcoholat reagent, including excluding addition of substoechiometric amounts of such alcoholat salt. The alcohol is a monovalent alcohol. The alkyl moiety R of such monovalent alcohols ROH may be branched or linear. Examples of such C1-C8 alcohol are methanol, ethanol, propanol, butanol, isobutanol, isopropanol, tert-butanol, hexanol, heptanol, octanol and the like, their constitutive isomers and mixtures thereof. More preferably, the alcohol is propanol, isopropanol, isobutanol or n-butanol. Most preferably, it is isopropanol.

‘Solid’ according to the present invention may be understood as powder, granules, pellets or the like. Suitable hydroxides can be any metal hydroxide, preferably, it is an alkaline earth or alkali metal hydroxide, most preferably it is sodium-, lithium- or potassium-hydroxide.

Another object of the present invention is a process for the preparation of imidocarbonic acid di-alkylesters of the formula III, 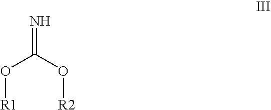
Wherein R1, R2 is alkyl, preferably symmetrically esterified imidocarbonic acid di-alkylesters of the formula III wherein R1 and R2 are the same, comprising the step of reacting cyanogen chloride ClCN with at least one secondary or tertiary C3-C5 alcohol ROH wherein R is R1 or R2 and wherein the alcohol encompasses in suspension a solid hydroxide.

A similar base-catalyzed reaction for preparation of imidates has been described (with nitroalkyl substituted cyanide: Schaefer et al., 1961, J. Org. Chem. 26:412; with cyanogen bromide: Lopyrev et al., 1989, Izvestiya Akademii Nauk SSSR, Seriya Khimicheskaya 10:2363, ISSN: 0002-3353); in the absence of an alcoholat, the synthesis described did not work for secondary or tertiary alkylalcohols whereas primary alcohols were found to be sufficient reactive in the absence of an alkoxide or alcoholat. Surprisingly, the use of cyanogen chloride in combination with solid, suspended hydroxide according to the present invention allowed of such reaction. Alcoholat salts are more expensive reagents and much less convenient to handle in particular at industrial scale operations due to their hygroscopic nature.

The above described preferred embodiments of the invention apply likewise to this other object of the invention where pertaining to the reaction of such cyanogenhalide. In particular, it is preferred that the alcohol is a C₃-C₅ alcohol, more preferably is isopropyl-alcohol. Further it is preferred that the reaction is carried out free from any addition of an alcoholat reagent as has been set forth above in more detail already.

### EXAMPLES

Possible embodiments for synthesis of 2-isopropoxy-6-(trifluoromethyl)pyrimidin-4-ol (4) according to the below reaction scheme I are given in the examples.

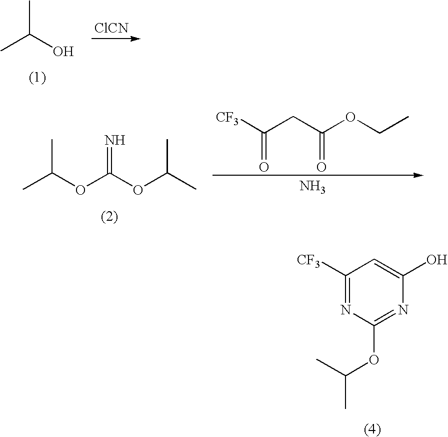

### Example 1

### Synthesis of imidocarbonic acid diisopropylester (2)

2 mol (123 g) of gaseous cyanogenclioride are injected in a suspension of isopropyl alcohol (720 g/12 mol) and solid NaOH pellets (838.3 g/2.21 mol) at 12-17° C. for 2.5 h under stirring. Thereafter the reaction is kept at 20° C. for further 3 hours. The mixture is then filtered in the cold at 5° C. to remove salt precipitate. The filtrate yields compound 2 in 86% yield but rather impure (23.3% by weight as determined by GC). Further distillation at 63° C. and 33 mbar results in pure compound 2 (98% by weight, with GC; cp. Matacz et al., 1988, Bulletin Polish Acad. Sci. Chemistry, 36:139 if; identity of product peak further checked with GC/MS, yielding mass peak m/z 62 for [C₄NO₂]⁺) at 74% yield (218.9 g). The crude filtrate is however sufficient for use as the educt in the next reaction step (s. example 3).

Pure product 2: ¹H-NMR(CCl4): 5.97 ppm (s,1H), 4.84 ppm (sept, 1H), 4.55 ppm (sept, 1H), 1.20 ppm (d,6H), 1.17 ppm (d, 6H)

### Example 2

### Synthesis of 2-isopropoxy-6-(trifluoromethyl)pyrimidin-4-ol (4)

14.87 g (1 eq./0.1 mol) of 98.5% pure compound 2 are mixed with 2.35 mol NH3 (25% in water) and 71.2 g (1.2 eq) of isopropyl alcohol at room temperature. 20.92 g (1.2 eq/0.11 mol) of trifuoromethylacetoacetic acid ethylester are stepwisely added thereto and the resulting mixture is stirred for 6.5 h at 60° C. and for further 2.5 h at 78° C. in an air-tight sealed reaction flask. The isopropyl alcohol is then largely removed by distillation as to obtain a yellowish, clear oil (about 80% content of compound 4) which oil is then transferred at about 45° C. into 10 times its volume of deionized water (200 ml). The product 4 immediately precipitates quantitatively. The precipitate is filtered off in the cold at 5° C. and is dried under vacuo. The resulting product is 98% pure as determined with HPLC. The analytical yield is 65%. Further 20% of product 4 at 88% purity may be obtained by slow crystallization from the residual filtrate in the cold and can be further recrystallized to give a 14% final yield (>98% by HPLC).

Product 4:

¹H-NMR(CCl4): 12.83 ppm (s, 1H), 6.43 ppm (s, 1H), 5.25 ppm (sept, 1H), 1.34 ppm (d, 6H).

### Example 3

### Synthesis of 2-isopropoxy-6-trifluoromethyl)pyrimidin-4-ol (4) in a One-Pot-Synthesis Approach

The synthesis is essentially carried out as described in examples 1+2, except that 124 g (0.21 mol/eq.) of the the crude, filtered product solution from example 1 (24% by weight) is employed as product 2 starting material for the reaction. Further, the reaction is carried out first at 2 h/60° C. and then 6 h/80° C.>98% pure product 4 is obtained at 65% yield. The overall yield including the reaction step of example 1 is 59%.

### Example 4

### Synthesis of 2-isopropoxy-6-(trifluoromethyl)pyrimidin-4-ol (4)

The synthesis is essentially carried out as described in example 3, except that 1.2 eq of trifluoromethylacetoacetic acid methylester were used. >97.7% pure product 4 is obtained at 67% yield.

### Example 5

### Synthesis of 2-isopropoxy-6-(trifluoromethyl)pyrimidin-4-ol (4)

The synthesis is essentially carried out as described in example 3, except that 1.2 eq of trifluoromethylacetoacetic acid isopropylester were used. >97% pure product 4 is obtained at 39% yield, but only after additional slow crystallisation of the crude product from isopropanol.

1. A process for preparing a 2-alkoxy-6-(trifluoromethyl)pyrimidine-4-ol of formula I: 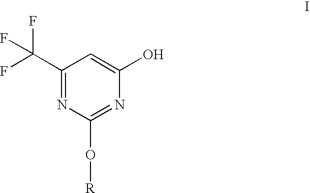
wherein R is C₁-C₈alkyl, comprising the steps of first reacting a cyanogen halide selected from the group consisting of cyanogen chloride ClCN and cyanogen bromide BrCN, with a C₁-C₈ alcohol ROH and/or a C₁-C₈ alcoholate in the presence of a base to give the corresponding symmetric imidocarbonic acid dialkylester of formula II: 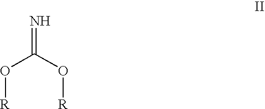
and, in a second step, further reacting said imidocarbonic acid dialkylester with trifluoromethyl acetoacetic acid ethyl ester to compound I in the presence of ammonia to yield the compound of formula I.

2. The process according to claim 1, wherein the process according to claim 1 is carried out as a one pot reaction.

3. The process according to claim 2, wherein the cyanogen halide is added to the reaction mixture at less than 20° C.

4. The process according to claim 3, wherein the product of formula II is obtained only by removing salt precipitates from the reaction broth of the first step.

5. The process according to claim 4, wherein the second reaction step is carried out with 1.5 to 3 mol equivalents of ammonia.

6. The process according to claim 5, wherein the second reaction step is carried out in an aprotic, polar solvent.

7. The process according to claim 6, wherein the second reaction step is carried out at a temperature of from 50 to 100° C.

8. The process according to claim 7, wherein the product compound of formula I is purified from the reaction sump by first removing the solvent and second crystallizing the compound of formula I from aqueous solution.

9. The process according to claim 8, wherein a C₁-C₈ alcohol is used.

10. The process according to claim 9, wherein the alcohol is isopropyl alcohol.

11. The process for the preparation of an imidocarbonic acid dialkylester of the formula III, wherein R¹ and R² are C₁-C₈ alkyl groups: 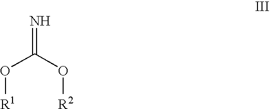
comprising the step of reacting cyanogen chloride ClCN with at least one C₁-C₈ alcohol ROH wherein R is R¹ or R² and wherein the alcohol encompasses in suspension a solid hydroxide.

12. The process according to claim 11, wherein the alcohol is a C₃-C₅ alcohol.

13. The process according to claim 11, wherein the reaction is carried out free from any addition of an alcoholate reagent.

14. A process for preparing a 2-alkoxy-6-(trifluoromethyl)pyrimidine-4-ol of formula I: 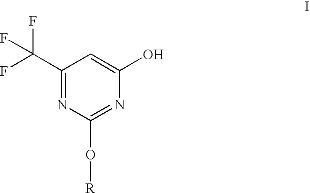
wherein R is C₁-C₈ alkyl, comprising the steps of first reacting a cyanogen halide selected from the group consisting of cyanogen chloride ClCN and cyanogen bromide BrCN, with a C₁-C₈ alcohol ROH and/or C₁-C₈ alcoholate in the presence of a base to give the corresponding symmetric imidocarbonic acid dialkylester of formula II: 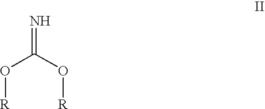
and, in a second step, reacting said imidocarbonic acid dialkylester with a trifluoromethyl acetoacetic acid alkyl ester, wherein the alkyl is C₁-C₆ alkyl, I in the presence of ammonia to yield the compound of formula I.

15. The process according to claim 1, wherein the cyanogen halide is added to the reaction mixture at less than 20° C.

16. The process according to claim 4, wherein the product of formula II is obtained only by removing salt precipitates from the reaction broth of the first step at a temperature of less than 10° C.

17. The process according to claim 7, wherein the second reaction step is carried out at a temperature of from 60 to 90° C.

18. The process according to claim 8, wherein the product compound of formula I is purified from the reaction sump by first removing the solvent and second crystallizing the compound of formula I from aqueous solution at an pH of from 5 to 7.

19. The process according to claim 9, wherein the alcohol is a C₃-C₅ alcohol.

20. The process according to claim 1, wherein the product of formula II is obtained only by removing salt precipitates from the reaction broth of the first step.

21. The process according to claim 1, wherein the product of formula II is obtained only by removing salt precipitates from the reaction broth of the first step at a temperature of less than 10° C.

22. The process according to claim 1, the second reaction step is carried out with 1.5 to 3 mol equivalents of ammonia.

23. The process according to claim 1, wherein the second reaction step is carried out in an aprotic, polar solvent.

24. The process according to claim 1, wherein the second reaction step is carried out at a temperature of from 50 to 100° C.

25. The process according to claim 1, wherein the second reaction steps is carried out at a temperature of from 60 to 90° C.

26. The process according to claim 1, wherein the product compound of formula I is purified from the reaction sump by first removing the solvent and second crystallizing the compound of formula I from aqueous solution.

27. The process according to claim 1, wherein the product compound of formula I is purified from the reaction sump by first removing the solvent and second crystallizing the compound of formula I from aqueous solution at an pH of from 5 to 7.

28. The process according to claim 1, wherein a C₁-C₈ alcohol is used.

29. The process according to claim 1, wherein an alcohol is a C₃-C₅ alcohol.

30. The process according to claim 29, wherein the alcohol is isopropyl alcohol.

31. The process according to claim 11, wherein the imidocarbonic acid dialkylester of formula III is a symmetrically esterified imidocarbonic acid dialkylester of the formula III wherein R¹=R².

32. The process according to claim 12, wherein the alcohol is isopropyl alcohol.

33. The process for the preparation of an imidocarbonic acid dialkylester of formula II, wherein R is C₁-C₈ alkyl: 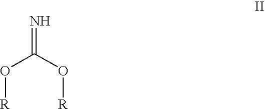
comprising the step of reacting cyanogens chloride ClCN with a C₁-C₈ alcohol ROH and wherein the alcohol encompasses in suspension a solid hydroxide.
